(19) United States
(12) Patent Application Publication
(10) Pub. No.: US 2014/0188156 A1
(43) Pub. Date: Jul. 3, 2014

(54) VENOUS CLOT BASKET

(71) Applicant: COOK MEDICAL TECHNOLOGIES LLC, BLOOMINGTON, IN (US)

(72) Inventors: Kurt J. Tekulve, Ellettsville, IN (US); Frank J. Fischer, JR., Bloomington, IN (US)

(73) Assignee: COOK MEDICAL TECHNOLOGIES LLC, BLOOMINGTON, IN (US)

(21) Appl. No.: 14/134,357

(22) Filed: Dec. 19, 2013

Related U.S. Application Data
(60) Provisional application No. 61/746,321, filed on Dec. 27, 2012.

(51) Int. Cl. A61B 17/221

(52) U.S. Cl. CPC A61B 17/221
USPC 606/200

(57) ABSTRACT

A system for removing a blood clot from a blood vessel or vein is provided. The system includes a plurality of support members that can be expanded to define a basket. The support members include a mesh portion at their distal ends. The system includes a first wire attached to the proximal end of the basket defined by the support members and a second wire attached to a distal end of the basket. The second wire extends through the middle of the basket when the basket is in the expanded condition. The width of the basket can be adjusted by adjusting the second wire relative to the first wire, and the location of the basket can be adjusted by adjusting the first wire. The basket is delivered distally beyond the blood clot, where it is expanded outward. The blood clot is captured by translating the basket proximally.

### CROSS REFERENCE TO RELATED APPLICATIONS

This application claims the benefit of U.S. Provisional Patent Application No. 61/746,321 filed Dec. 27, 2012, the content of which is hereby incorporated by reference in its entirety.

### BACKGROUND

The present embodiments relate generally to a system, apparatus, and method for closing removing a clot from the venous system and, more particularly, for an expandable and adjustable basket for removing a blood clot from the blood vessel.

A venous system includes myriad blood vessels for transferred and circulating blood throughout the human body. Blood clots can form throughout the venous system over time, which can result in a dangerous condition for the patient depending on the size and/or location of the clot. The blood clots that form inside a blood vessel can be referred to as a thrombosis.

One type of thrombosis is known as Deep Vein Thrombosis or Deep Venous Thrombosis and is commonly abbreviated as “DVT.” DVT is the formation of a blood clot in a deep vein, as can commonly occur in the veins within a patient's legs. DVT can result in substantial discomfort to patient, as well as affecting the external appearance of the patient's legs. As such, DVT can result in pain, swelling, redness, or engorged superficial veins. Additionally, DVT can lead to a potentially life threatening condition, known as a pulmonary embolism, where a blood clot can detach from a vein and travel to the lungs. Thus, it is desirable to limit the occurrence of DVT or remove the clots from the veins.

Thus, there is a need for a device that can effectively and reliably remove a blood clot from a patient's blood vessel.

### SUMMARY

A system for removing a blood clot from a blood vessel is provided, the system comprising: a tube member having a proximal end and a distal end, the tube member having a lumen formed through the proximal and distal ends, the lumen defining a longitudinal axis therealong; a first support member having a proximal end and a distal end, the first support member having a first mesh portion at the distal end thereof; a second support member having a proximal end and a distal end, the second support member having a second mesh portion at the distal end thereof; a first wire extending through the lumen, the first wire being attached to the proximal ends of the first and second support members; a second wire extending through the lumen, the second wire being attached to the distal ends of the first and second support members; wherein the first and second support members define a basket therebetween when the first and second support members are expanded from the second wire; wherein the second wire is configured for adjusting the basket between an expanded state and a compressed state; wherein the first wire is configured for adjusting the longitudinal location of the basket; and wherein the second wire extends within the basket defined by the expanded first and second support members when the basket is in the expanded state.

In another form, the longitudinal location of the basket is adjusted proximally by pulling on the first wire relative to the tube member.

In another form, the support members are made from a shape memory material.

In another form, the support members are configured to expand into the expanded condition based on the shape memory material.

In another form, the support members are configured to expand into a further expanded condition by pulling on the second wire relative to the first wire.

In another form, the expanded condition of the basket is maintained by pulling on both the first wire and the second wire.

In another form, the expanded condition of the basket is narrowed by pulling on the first wire relative to the second wire.

In yet another form, a method for removing a blood clot from a blood vessel is provided, the method comprising the steps of: inserting a tube member having a first support member and a second support member disposed therein to a location of a blood clot within a blood vessel, wherein the support members have proximal and distal ends thereof, a first wire is attached to the proximal end of the support members, and a second wire is attached to the distal end of the support members; extending the support members out of the tube member, wherein the distal ends of the support members are exposed; locating the distal ends of the support members distally from a blood clot; expanding the support members outwardly from the second wire to form a basket having proximal and distal ends thereof, wherein at least a portion of the second wire is disposed within the basket and radially between the support members; and withdrawing the basket proximally to remove the blood clot.

In another form, the support members are made from a shape memory material, and the shape memory material expands the support members to form the basket.

In another form, the support members include a mesh portion at distal ends thereof, and the mesh portions form the basket in response to expanding the support members.

In another form, the method further comprises additionally expanding the support members to contact the vessel wall.

In another form, the method further comprises moving the second wire proximally relative to the first wire to expand the support members.

In another form, the method further comprises moving the first wire proximally to pull the proximal end of the basket toward the tube member.

In another form, the method further comprises moving the first wire and second wire together to maintain the shape of the basket member and adjust the longitudinal location of the basket.

In yet another form, a blood clot retrieval apparatus is provided comprising: a first support member having a proximal end and a distal end, the first arm member having a first mesh portion at the distal end thereof; a second support member having a proximal end and a distal end, the second arm member having a second mesh portion at the distal end thereof; a first wire extending proximally and attached to the proximal ends of the first and second support members; a second wire extending proximally and attached to the distal ends of the first and second support members; wherein the first and second support members are configured to adjust between a compressed condition and an expanded condition; wherein the expanded condition defines a basket; wherein the second wire extends within the basket; and wherein the second wire is configured for adjusting the width of the basket.

In another form, the distal ends of first and second support members are mounted within a distal housing.

In another form, the apparatus further comprises a wire guide coil attached at the distal ends of the first and second support members.

In another form, the first and second wire are adjustable relative to each other to change the shape of the basket defined by the first and second support members.

In another form, the first and second wire are adjustable together to maintain the shape of the basket defined by the first and second support members while translating the basket.

In another form, the support members are made from a material having shape memory characteristics that define a shape of the basket absent actuation by the first or second wires.

### BRIEF DESCRIPTION OF THE DRAWINGS

FIG. 1A is a schematic view of a system for removing a clot from a blood vessel;

FIG. 1B is a schematic view of the system showing a handle;

FIG. 2 is a side view of a distal end of a basket defined by support members;

FIG. 3 is a side view of a proximal end of the basket;

FIG. 4 is a schematic view of the system showing the basket being delivered distally beyond a blood clot;

FIG. 5 is a schematic view of the basket in an expanded condition;

FIG. 6 is a schematic view of the basket in another expanded condition; and

FIG. 7 is a schematic view of the basket having the blood clot captured therein.

### DETAILED DESCRIPTION

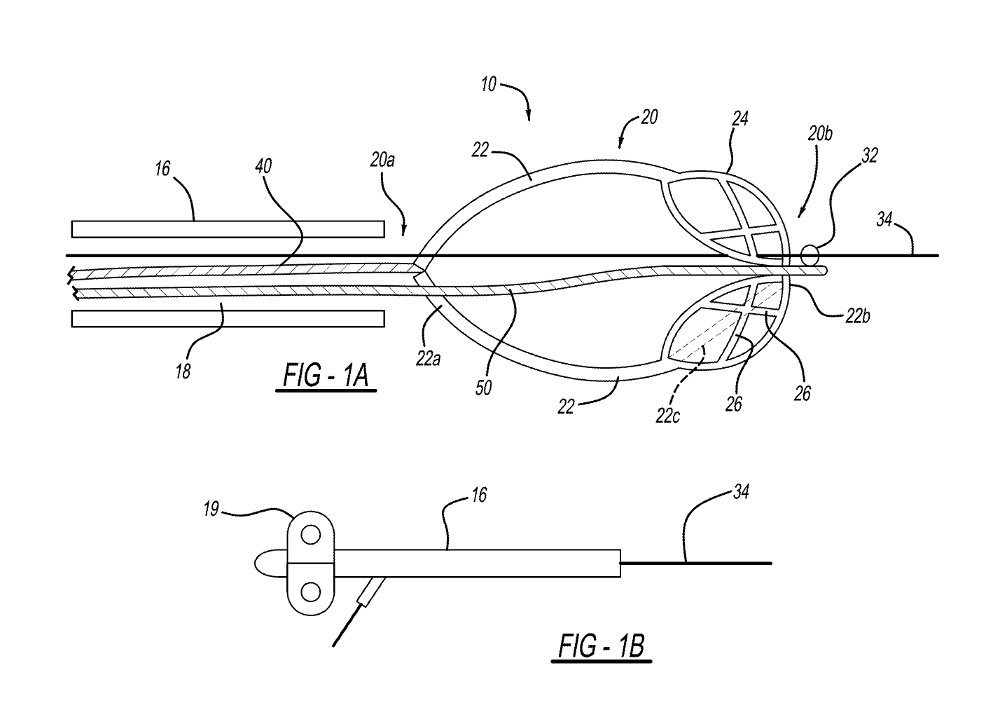

Referring now to the drawings, FIGS. 1A-7 illustrate a system 10 for removing a blood clot 12 from a blood vessel 14. The system 10 includes a tube member or catheter 16 having a generally tubular shape and a lumen 18 extending therealong. A handle 19 can be attached to the proximal end of the catheter 16 for controlling the system. The system further includes a collapsible basket 20 that is configured to transfer from a compressed condition into an expanded condition. The basket 20 can be housed within the catheter 16 for delivery to the location of the blood clot 12, as further described below.

With reference to FIG. 1A, the basket 20 includes a plurality of support members 22 extending from a proximal end 20a of the basket 20 to the distal end 20b of the basket 20. In one form, the basket 20 includes two support members 22; however, other quantities of support members 22 can also be used, such as a basket formed from four support members 22. For purposes of discussion, a pair of support members 22 will be described. Each support member 22 has a generally elongate shape with a proximal end 22a and a distal end 22b. The support members 22 can be made from a material having shape memory characteristics, such as Nitinol, such that the support members 22 will tend to spring outward from a compressed condition to expand the basket 20. Alternatively, the shape memory characteristics could be tuned to another shape, such as the compressed condition, if desired.

Each support member 22 also includes a mesh portion 24 that is disposed at the distal end thereof. The mesh portion 24 can be formed from a plurality of interconnected struts 26 that are collectively mounted to the support member 22. The mesh portion 24 can be made from the same material as the support member 22, such that the mesh portion 24 is integrally formed with the support member 22. Alternatively, the struts 26 of the mesh portion 24 can be attached as separate pieces of the support member 22.

The size of the mesh portion 224 can vary depending on the needs of the user and size of the blood vessel 14. Additionally, the space between the struts 26 can be varied by adding or subtracting struts 26 to the mesh portion 24 to accommodate various blood clot 12 sizes. Thus, it will be appreciated that the mesh portion 24 is scalable in this regard depending on the needs of the user.

In one form, the support member 22 can include a main support strut 22c (shown in broken line in FIG. 1A) that extends from the proximal end 20a to the distal end 20b of the basket 20, with the struts 26 of the mesh 24 extending from the main support strut 22c. In another form, the struts 26 can form the mesh 24, with the length of the support member 22 being interrupted by the mesh 24, such that the support member 22 is effectively broken up, with the distal end 22b defined by the mesh 24, and the proximal end 22a of the support member 22 extending proximally from the mesh 24. The mesh 24 can be made from Nitinol or another material having shape memory characteristics, such that mesh 24 will open outwardly upon being released from a compressed condition.

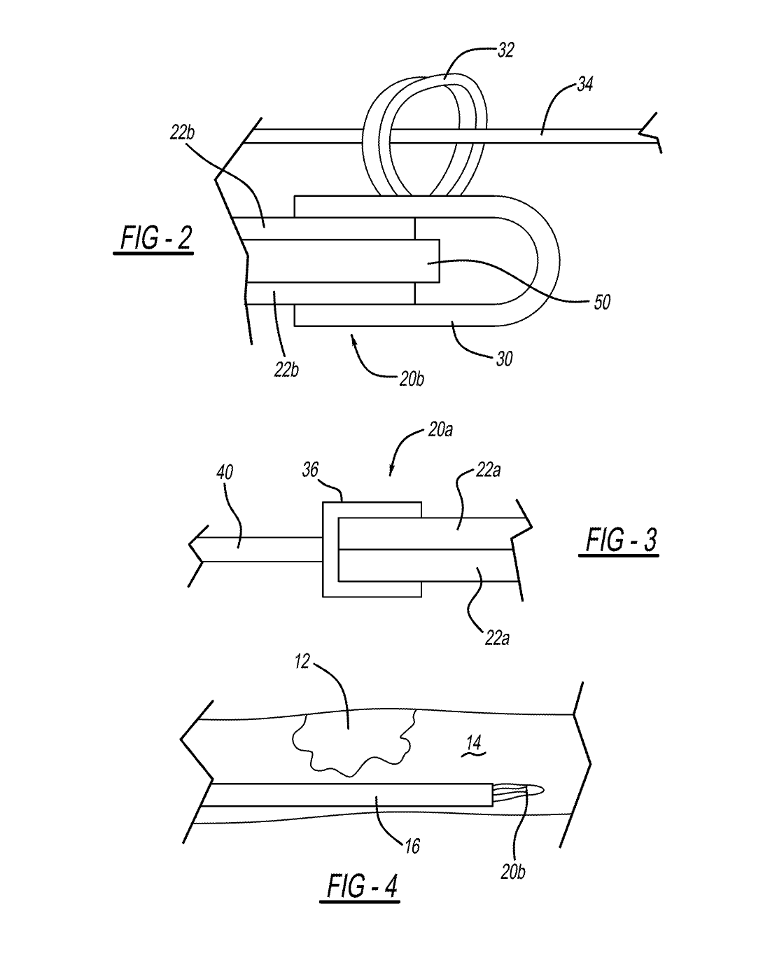

With reference to FIG. 2, the distal ends 22b of the support members 22 can be mounted together at the distal end 20b of the basket 20. The support members 22 can be mounted together via a distal tip housing 30 that encompasses the distal ends 22b of the support members 22. The distal tip housing 30 can further include a wire guide coil 32 in the form of a loop extending therefrom. However, in another form, the wire guide coil could be formed by one or more of the support members 22. The wire guide coil 32 is configured for allowing a guidewire 34 to be inserted therethrough for assisting in locating the basket 20 along the venous system.

With reference to FIG. 3, the proximal ends 22a of the support members 22 can be mounted together at the proximal end 20a of the basket 20 as well. In one form a proximal tip housing 36 can encompass the proximal ends of the support members 22 22 similar to the distal tip housing 30 described above. In another form, the proximal ends 22a can be fused together in a manner known in the art. In another form, the proximal ends 22a can be banded together. In another form, the proximal ends 22a can be wound or braided together. Of course, other manners of joining the proximal ends 22a of the support members 22 at the proximal end 20a of the basket 20 can also be used.

The system 10 further includes a first wire 40 member mounted to the proximal end 20a of the basket 20. The first wire 40 can generally extend through the lumen 18 to the handle 19 so that the wire can be actuated and adjusted by the user at the handle 19. The first wire 40 is configured for adjusting the longitudinal position of the basket 20. More specifically, the first wire 40 is configured to be pulled for translating the basket 20 back toward the tube or catheter 16 after the basket 20 has been deployed. The distal end of the first wire 40 can be mounted or attached to the proximal ends 22a of the support members 22 via known methods. In one form, the first wire 40 can be in the form of the support members 22 wound or braided together to form the first wire 40.

With reference again to FIG. 2, in addition to the first wire 40, the system 10 includes a second wire 50 that is mounted to the distal ends of the support members 22 and the basket 20. The second wire 50, like the first wire 40, extends through the lumen 18 and is attached or mounted to the handle 19 so that it can be actuated or adjusted by the user at the handle 19. The second wire 50, unlike the first wire 40, extends along the length of the support members 22 due to its mounting at the distal ends 22b thereof. The second wire 50 will extend within the support members 22 when the support members 22 are expanded outward to define the expanded basket 20 when the basket 20 has been deployed from the tube or catheter 16. The second wire 50 is configured to adjust the expanded shape of the basket 20 defined by the support members 22 and mesh portions 24. More specifically, the second wire 50 can be pulled to move the distal end 20b of the basket 20 to cause the basket 20 to compress longitudinally, thereby causing it to expand radially. The second wire 50, being attached to the distal ends of the support members 22 and the basket 20, can be mounted within the distal tip housing 30 along with the distal ends of the support members 22. However, the second wire 50 could also be mounted outside the housing 30 or otherwise near the distal ends of the support members 22 and basket 20.

The first wire 40 and the second wire 50 are preferably adjustable independent of each other to control the shape and location of the basket 20. In one form, the wires 40 and 50 can have a generally rigid form to allow the wires 40 and 50 to be both pulled and pushed from the handle 19 to adjust the longitudinal location of either the proximal end of the basket 20 or the distal end of the basket 20, or both. Pulling on the first wire 40 relative to the second wire 50 will cause the basket 20 to become longer and narrower. Pulling on the second wire 50 relative to the first wire 40 will cause the basket 20 to become shorter and wider. Pulling on both wires 40 and 50 will cause the basket 20 shape to remain relatively constant while pulling the basket 20 proximally. It will be appreciated that the wires 40 and 50 can likewise be pushed relative to each other, or together, to effect similar changes in the basket 20 shape and location.

The system further includes the guidewire 34, mentioned above with respect to the wire guide coil. The guidewire 34 can extend through the lumen 18 of the catheter 16 along with the first and second wires 40 and 50. Similar to the second wire 50, the guidewire 34 will extend along the length of the basket 20 and can extend within the basket 20 when the basket 20 is expanded. The guidewire 34 allows the catheter 16 and the basket 20 housed therein to be delivered to the area of the blood clot 12 within the blood vessel 14.

Having described the structure of the system 10 above, the function and use of the system 10 will now be described.

To remove a blood clot 12 from within a blood vessel 14, the surgeon can identify the location of the blood clot 12 according to known medical methods. The tube or catheter 16, having the support members 22 that form the basket 20 contained within the lumen 18 in a compressed condition, can be inserted into the blood vessel 14 via a percutaneous procedure or other minimally invasive method known in the art.

With reference to FIG. 4, the tube or catheter 16 can be guided along the guidewire 34 to the location of the blood clot 12 within the vessel 14. Once at the location of the blood clot 12, the catheter 16 can be inserted past the blood clot 12, where the catheter 16 can then be retracted to expose the support members 22 forming the basket 20 in the compressed condition. Alternatively, the basket 20 can be pushed out from the catheter 16, with the catheter 16 remaining stationary to expose the support members 22 forming the compressed basket 20.

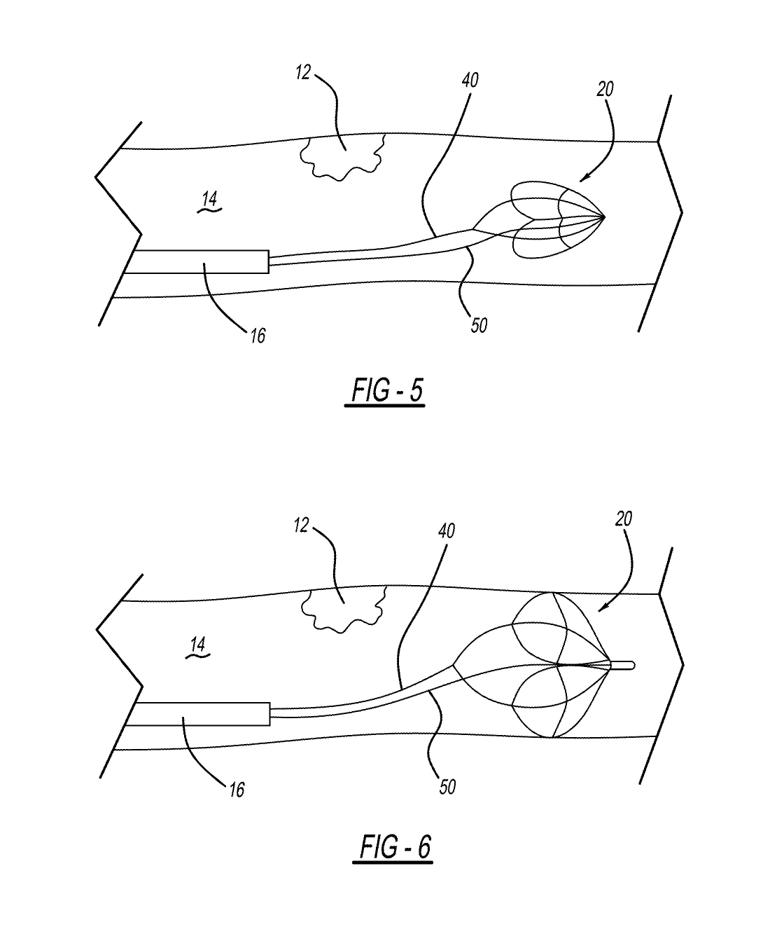

With reference to FIG. 5, once the support members 22 are exposed, the shape memory characteristics of the support members 22 will cause the support members 22 to expand outwardly to form the expanded basket 20. More specifically, the mesh portion 24s of the support members 22 will expand outwardly as well. The expanded condition caused by the shape memory characteristics of the support members 22 can be an intermediate expanded condition, where the basket 20 does not fully expand to span the width of the blood vessel 14, depending on the size of the basket 20 determined by the shape memory characteristics relative to the size of the blood vessel 14.

With the basket 20 in this intermediate condition, the second wire 50 can be adjusted proximally, or pulled, to cause the distal end of the basket 20 to translate proximally relative to the proximal end of the basket 20. This relative translation will cause the basket 20 to expand into a fully expanded condition that extends across substantially the entire width of the blood vessel 14, as shown in FIG. 6. It will be appreciated that the reference to the fully expanded condition does not necessarily mean the maximum amount of radial expansion, but rather the desired expanded width of the basket 20 as determined by the user.

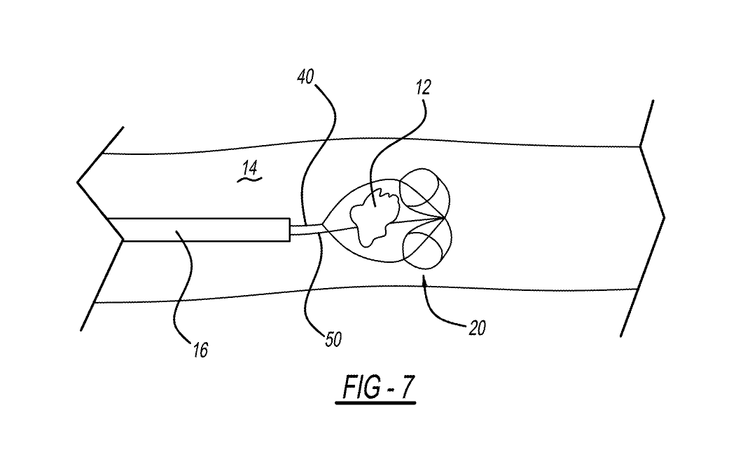

With reference to FIG. 7, with the basket 20 in the desired expanded condition, the first and second wire 50 can be pulled proximally to translate the basket 20 proximally and along the blood vessel 14 toward the clot 12. The basket 20 can then capture the blood clot 12 or embolism (or multiple clots 12 or emboli) as the basket 20 translates.

The basket 20 can be further translated proximally toward the catheter 16, delivering the blood clot 12 or embolism toward the catheter 16 as well. Once the blood clot 12 has been delivered to the catheter 16, the clot 12 can be aspirated out through the catheter 16. In another form, the entire system 10 can be pulled back with the clot 12 captured by the basket 20 to remove the blood clot 12 as the catheter 16 and basket 20 are removed from the blood vessel 14.

Alternatively, or in addition to the above described method, the blood clot 12 can be broken up or compressed by the basket 20. As previously described, the shape of the basket 20 can be adjusted by adjusting the first and second wire 50s relative to each other. With the blood clot 12 captured within the basket 20, the basket 20 can be compressed by moving the first wire 40 proximally relative to the second wire 50 to extend the length of the basket 20 and narrow the width of the basket 20, thereby applying a compressive force to the clot 12 to break up or compress the clot 12 to assist in aspirating the clot 12 through the catheter 16.

At the conclusion of the clot 12 retrieval, the system 10 can be removed from the blood vessel 14, completing this aspect of the medical procedure. The surgeon can subsequently perform additional procedures or complete the surgical intervention in a manner known in the art.

As a person skilled in the art will readily appreciate, the above description is meant as an illustration of implementation of the principles this invention. This description is not intended to limit the scope or application of this invention in that the invention is susceptible to modification, variation, and change, without departing from the spirit of this invention, as defined in the following claims.

What is claimed is:

1. A system for removing a blood clot from a blood vessel, the system comprising:
a tube member having a proximal end and a distal end, the tube member having a lumen formed through the proximal and distal ends, the lumen defining a longitudinal axis therealong;
a first support member having a proximal end and a distal end, the first support member having a first mesh portion at the distal end thereof;
a second support member having a proximal end and a distal end, the second support member having a second mesh portion at the distal end thereof;
a first wire extending through the lumen, the first wire being attached to the proximal ends of the first and second support members;
a second wire extending through the lumen, the second wire being attached to the distal ends of the first and second support members;
wherein the first and second support members define a basket therebetween when the first and second support members are expanded from the second wire;
wherein the second wire is configured for adjusting the basket between an expanded state and a compressed state;
wherein the first wire is configured for adjusting the longitudinal location of the basket; and
wherein the second wire extends within the basket defined by the expanded first and second support members when the basket is in the expanded state.

2. The apparatus of claim 1, wherein the longitudinal location of the basket is adjusted proximally by pulling on the first wire relative to the tube member.

3. The apparatus of claim 1, wherein the support members are made from a shape memory material.

4. The apparatus of claim 3, wherein the support members are configured to expand into the expanded condition based on the shape memory material.

5. The apparatus of claim 4, wherein the support members are configured to expand into a further expanded condition by pulling on the second wire relative to the first wire.

6. The apparatus of claim 1, wherein the expanded condition of the basket is maintained by pulling on both the first wire and the second wire.

7. The apparatus of claim 1, wherein the expanded condition of the basket is narrowed by pulling on the first wire relative to the second wire.

8. A method for removing a blood clot from a blood vessel, the method comprising the steps of:
inserting a tube member having a first support member and a second support member disposed therein to a location of a blood clot within a blood vessel, wherein the support members have proximal and distal ends thereof, a first wire is attached to the proximal end of the support members, and a second wire is attached to the distal end of the support members;
extending the support members out of the tube member, wherein the distal ends of the support members are exposed;
locating the distal ends of the support members distally from a blood clot;
expanding the support members outwardly from the second wire to form a basket having proximal and distal ends thereof, wherein at least a portion of the second wire is disposed within the basket and radially between the support members; and
withdrawing the basket proximally to remove the blood clot.

9. The method of claim 8, wherein the support members are made from a shape memory material, and the shape memory material expands the support members to form the basket.

10. The method of claim 8, wherein the support members include a mesh portion at distal ends thereof, and the mesh portions form the basket in response to expanding the support members.

11. The method of claim 8 further comprising additionally expanding the support members to contact the vessel wall.

12. The method of claim 8 further comprising moving the second wire proximally relative to the first wire to expand the support members.

13. The method of claim 8 further comprising moving the first wire proximally to pull the proximal end of the basket toward the tube member.

14. The method of claim 8 further comprising moving the first wire and second wire together to maintain the shape of the basket member and adjust the longitudinal location of the basket.

15. A blood clot retrieval apparatus comprising:
a first support member having a proximal end and a distal end, the first arm member having a first mesh portion at the distal end thereof;
a second support member having a proximal end and a distal end, the second arm member having a second mesh portion at the distal end thereof;
a first wire extending proximally and attached to the proximal ends of the first and second support members;
a second wire extending proximally and attached to the distal ends of the first and second support members;
wherein the first and second support members are configured to adjust between a compressed condition and an expanded condition;
wherein the expanded condition defines a basket;
wherein the second wire extends within the basket; and
wherein the second wire is configured for adjusting the width of the basket.

16. The apparatus of claim 15, wherein the distal ends of first and second support members are mounted within a distal housing.

17. The apparatus of claim 15 further comprising a wire guide coil attached at the distal ends of the first and second support members.

18. The apparatus of claim 15, wherein the first and second wire are adjustable relative to each other to change the shape of the basket defined by the first and second support members.

19. The apparatus of claim 15, wherein the first and second wire are adjustable together to maintain the shape of the basket defined by the first and second support members while translating the basket.

20. The apparatus of claim 15, wherein the support members are made from a material having shape memory characteristics that define a shape of the basket absent actuation by the first or second wires.
